(19) United States
(12) Patent Application Publication
(10) Pub. No.: US 2025/0057565 A1
(43) Pub. Date: Feb. 20, 2025

(54) CATHETER DEVICE FOR EXTRACTING AN OCCLUSION FROM A BLOOD VESSEL

(71) Applicant: CLEARSTREAM TECHNOLOGIES LIMITED, Enniscorthy, County Wexford (IE)

(72) Inventors: Seán Wall, Enniscorthy, County Wexford (IE); Pat Byrne, Enniscorthy, County Wexford (IE); Derek Doyle, Enniscorthy, County Wexford (IE)

(21) Appl. No.: 18/720,992

(22) PCT Filed: Dec. 17, 2021
(86) PCT No.: PCT/EP2021/086543

§ 371 Date: Jun. 17, 2024

(51) Int. Cl. A61B 17/3207; A61B 17/00

(52) U.S. Cl. CPC A61B 17/320725; A61B 17/00234; A61B 2017/00238; A61B 2017/00305; A61B 2217/005; A61B 2217/007

(57) ABSTRACT

A catheter device for extracting an occlusion from a blood vessel includes a shaft having a proximal end and a distal end; and an expandable structure (3) having a proximal portion and a distal portion and coupled to or adjacent the distal end of the shaft; the expandable structure being configured to expand radially around the shaft; characterised in that the proximal portion of the expandable structure comprises one or more fragmentors (4, 5).

### TECHNICAL FIELD

This disclosure relates to a catheter device for extracting an occlusion from a blood vessel, such as a thrombectomy device, and to a corresponding catheter method and system.

### BACKGROUND

Many vascular system problems stem from a disruption of normal blood flow through blood vessels. This insufficient or irregular blood flow may be caused by a blockage within a blood vessel referred to as a thrombus or a blood clot. Blood clots may cause conditions, some life-threatening, such as deep vein thrombosis, pulmonary embolism, acute stroke and acute myocardial infarction.

Thrombectomy is a procedure used to remove blood clots from arteries and veins, and non-invasive catheter-based thrombectomy devices have been developed based on various removal mechanisms. Typically, a catheter is inserted in the circular system of the patient and to the relevant blood vessel. A thrombectomy device is advanced through the catheter to the occluding blood clot. While some clots may be removed merely by suction, others may be too voluminous or too solidified for aspiration and must in a first instance be broken down to enable removal. Various techniques are known which break down or fragment blot clots. The clot may be broken down by advancing a coring or cutting device through the clot. Alternatively, an expandable device may penetrate the clot and expand whilst in the clot to break it from the inside. In these techniques, the fragmentation occurs from the proximal end or from the middle of the clot, so that clot fragments may break off from the distal end of the clot and, if not caught, may travel along the blood stream. These clot fragments (or emboli) may result in a multitude of new occlusions downstream of the original thrombus.

It is an object of this invention to mitigate problems such as those described above and to provide an improved alternative to existing processes and devices.

### SUMMARY

According to a first aspect of the invention, there is provided a catheter device for extracting an occlusion from a blood vessel, said device comprising a shaft having a proximal end and a distal end; and an expandable structure having a proximal portion and a distal portion and coupled to or adjacent the distal end of the shaft; said the expandable structure being configured to expand radially around the shaft; characterised in that the proximal portion of the expandable structure comprises one or more fragmentors for fragmenting the occlusion.

Within the context of the present invention, “proximal end” refers to the end closest to the medical practitioner or to the point of entry of the catheter device into the patient's blood vessel; and “distal end” refers to the end furthest to the medical practitioner or to the point of entry of the catheter device into the patient's blood vessel.

The proximal portion of the catheter device according to the disclosure comprises means for fragmenting (fragmentors) the occlusion, starting from the distal end of the occlusion. The distal end of the shaft comprising the expandable structure is positioned adjacent the proximal end of the occlusion. With the expandable structure in an unexpanded configuration, the shaft penetrates the occlusion, and is advanced through the occlusion. Once the proximal portion of the expandable structure has exited the occlusion, the expandable structure is expanded into a working configuration. The proximal portion of the expandable structure fragments the occlusion (from its distal end to its proximal end) as the catheter device is pulled back towards the occlusion. As the device is located downstream of the occlusion during the fragmentation step, any emboli breaking off from the occlusion may be caught by the device. Thus, the risk of losing fragmentation debris, and hence of subsequent embolization or thrombosis, is minimised.

As used herein, the term “means” can be equivalently expressed as, or substituted with, any of the following terms: device, apparatus, structure, part, sub-part, assembly, sub-assembly, machine, mechanism, article, medium, material, appliance, equipment, system, body or similar wording.

Within the context of the present disclosure, the term “occlusion” designates any material blocking or closing a blood vessel or hindering normal blood flow, and includes, but is not limited to a thrombus, a blood clot, an embolus or the like.

In one embodiment, the expandable structure is configured and arranged to expand or deploy from a first non-expanded configuration (e.g. a delivery, insertion and/or a retrieval configuration), in which the structure is substantially aligned or folded along the shaft, to a second expanded or deployed configuration (e.g. a working, fragmenting and/or collecting configuration), in which the structure is substantially aligned with the inner walls of the blood vessel.

The proximal portion may be tapered, or substantially conical. This configuration assists the penetration of the fragmentors, and hence of the catheter device, into the occlusion and the subsequent fragmentation of the occlusion. This feature is also advantageous during the process of retrieving the catheter device by facilitating the folding and retraction of the expandable structure into the catheter.

In one embodiment, the expandable structure comprises an intermediate portion located between the proximal portion and the distal portion.

In an embodiment, the proximal portion comprises a plurality of tethers. The proximal ends of the tethers may be coupled to the shaft of the device and the distal ends of the tethers are coupled to the intermediate portion of the expandable structure. This configuration is advantageous in that occlusion fragments may pass through the spaces between the tethers for subsequent collection. Alternative materials and structures (for example a mesh structure) are envisaged which allow passage of the fragments.

The tethers may also assist in the deployment and/or folding of the expandable structure.

Any fragmenting structure is envisaged which enables breaking down the occlusion into small fragments to facilitate the extraction of the occlusion from the blood vessel. In an embodiment, the one or more fragmentors comprise cutters for cutting the occlusion. The cutters may comprise one or more blades.

In an embodiment, the fragmentors are aligned with the outer contour of the proximal portion of the expandable structure, in order to provide direct cutting efficiency. For example, in the case of a conical proximal portion, the fragmentors (e.g. blades) may be arranged in a corresponding conical fashion on and/or along the proximal portion; in the case of tethers, the fragmentors (e.g. blades) may be arranged on and/or along the tethers.

The catheter device may comprise a lumen for fluid flow and fragment passage, said lumen extending at least between the proximal portion and the distal portion of the expandable structure. The fragmentors may be configured and arranged to guide occlusion fragments towards the lumen.

The fragmentors may be configured and arranged so as to guide the occlusion fragment towards and into the lumen. For example, the fragmentors may be selectively angled or profiled, or preferentially oriented. The expandable structure may comprise one or more chamfers extending partially or completely along the proximal portion. The fragmentors may be spiral-shaped or comprise spiral cutting elements.

In an embodiment, the expandable structure comprises an expandable scaffold. The expandable scaffold may comprise a substantially rigid material. The expandable scaffold may comprise a polymeric material, or more preferably a metallic material.

The expandable scaffold may be integrally formed or may comprise an assembly of components. The expandable scaffold may comprise a plurality of tethers, a mesh and/or a spiral.

In an embodiment, the expandable structure comprises a cover membrane. The cover membrane may consist of or comprise fibers, for example a high tensile strength fiber-based cover material.

The cover membrane may cover partially or completely the intermediate portion of the expandable structure. When covering the intermediate portion, the membrane assists in retaining the emboli and fragments within the lumen of the expandable structure. Additionally or alternatively, the cover membrane may cover partially or completely the distal portion of the expandable structure, so as to safely collect emboli and fragments. The combination of the expandable structure with a cover membrane advantageously provides a lower profile clot retrieval and collection system.

In an embodiment, the expandable structure comprises one or more inflatable balloons. For example, the structure may comprise one inflatable balloon surrounding the intermediate portion of the expandable structure. Alternatively, the structure may comprise a plurality of balloons, preferably configured and arranged so as to surround the lumen of the expandable structure. The one or more balloons may consist of or comprise fibers, for example a high tensile strength fiber-based material, such as UHMWPE.

In an embodiment, the balloons are inflatable radially outwards relative to the expandable structure. “radially outwards” may be relative to the intermediate portion of the expandable structure, or specific components of the expandable structure, such as individual components of the expandable scaffold or individual tethers.

In an embodiment, the distal portion of the expandable structure comprises one or more collecting elements for collecting the occlusion fragments. This feature is particularly advantageous to prevent the dissemination of emboli and fragments downstream of (distally from) the original occlusion, thereby minimising the risk of formation of subsequent occlusions. Within the context of the present invention, “downstream” refers to the distal side and “upstream” to the proximal side.

The distal portion of the expandable structure may be tapered or conical. This feature facilitates the collection of emboli and fragments.

The distal portion of the expandable structure may comprise tethers. The proximal ends of the tethers may be coupled to the distal end of the intermediate portion of the expandable structure, and the distal ends of the tethers are coupled to the shaft. This configuration is advantageous in that it assists in the deployment and folding of the expandable structure.

In an embodiment, the distal portion of the expandable structure comprises a basket for collecting emboli and fragments. The basket may be fluid permeable. For example, the basket may collect emboli and fragments, but allow blood flow therethrough. This configuration allows the extraction to be carried out, without preventing healthy blood and plasma from flowing through the blood vessel. The basket may consist of or comprise fibers, for example a high tensile strength fiber-based cover material. The basket may consist of or comprise a material or structure (such as a mesh) capable to allowing the passage of blood therethrough, whilst retaining fragments and particles. The basket may consist of or comprise a porous material and/or or a polymeric material. The polymeric material may be polyethylene-based, for example a ultrahigh molecular weight polyethylene, and/or a hybrid polymeric blend with one more monomers, such as polyurethane or a polyamide. The basket may consist of or comprise fibers, such as interwoven or electrospun fibers (e.g. large porosity electrospun fibers).

According to a second aspect of the invention, there is provided a catheter method for extracting an occlusion from a blood vessel, comprising the steps of advancing a catheter along the vessel; advancing a catheter device as described hereinabove through the catheter; advancing the device through the occlusion until the proximal portion of the device protrudes partially or completely beyond the occlusion; expanding the expandable structure; and retrieving the device so as to fragment the occlusion.

The catheter may be a delivery catheter or sheath. The catheter may be advanced through and along the circulatory system of the patient to the target blood vessel, until the distal end of the catheter is positioned adjacent the proximal end of the occlusion.

The catheter device may be advanced through and along the catheter and to the distal end of the catheter.

The shaft may penetrate the proximal end of the occlusion, together with the unexpanded expandable structure, and extend beyond the distal end of the occlusion until the proximal portion of the expandable structure has partially or completely exited the occlusion. The expandable structure may then be deployed so as to fragment the distal end of the obstructive material using the proximal portion of the expandable structure.

In an embodiment, the device is advanced through the catheter and/or through the occlusion, with the expandable structure is a non-expanded configuration.

In an embodiment, the step of expanding expandable structure comprises deploying an expandable scaffold.

In an embodiment, the step of expanding expandable structure comprises inflating a plurality of inflatable balloons.

In an embodiment, the method comprises the step of guiding the occlusion fragments towards and/or into the lumen of the expandable structure, for example by means of profiled, oriented and/or chamfered fragmentors.

In an embodiment, the catheter method further comprises the step of collecting the occlusion fragments, for example by means of a basket.

In an embodiment, the catheter method further comprises the step of retrieving the device through the occlusion and/or through the catheter, with the expandable structure is a non-expanded configuration.

According to a third aspect of the invention, there is provided a catheter system comprising a catheter and a catheter device as described hereinabove.

In an embodiment, the system further comprises one or more capturing elements for capturing the occlusion fragments. For example, the system may comprise one or more capturing element (such as a basket) for capturing the fragments collected in the lumen and/or the collection element of the expandable structure. The capturing element may be coupled to the catheter device or to the catheter, may be separately provided.

Additionally or alternatively, the system may comprise a suction system for aspirating the occlusion fragments.

According to further aspects of the invention, there are provided methods for the treatment of conditions requiring the removal of an occlusion from a blood vessel, such as deep vein thrombosis, pulmonary embolism, acute stroke and acute myocardial infarction.

### BRIEF DESCRIPTION OF THE DRAWINGS

The invention will be further described with reference to the drawings and figures, in which

FIG. 1 is a schematic representation of a catheter device according to the present invention;

FIGS. 2A to 2D are schematic illustrations of fragmentors in a catheter device according to the present invention;

FIGS. 3A to 3D are schematic partial illustrations of fragmentors in a catheter device according to the present invention; and

FIG. 4 is a schematic representation of another catheter device according to the present invention.

### DETAILED DESCRIPTION

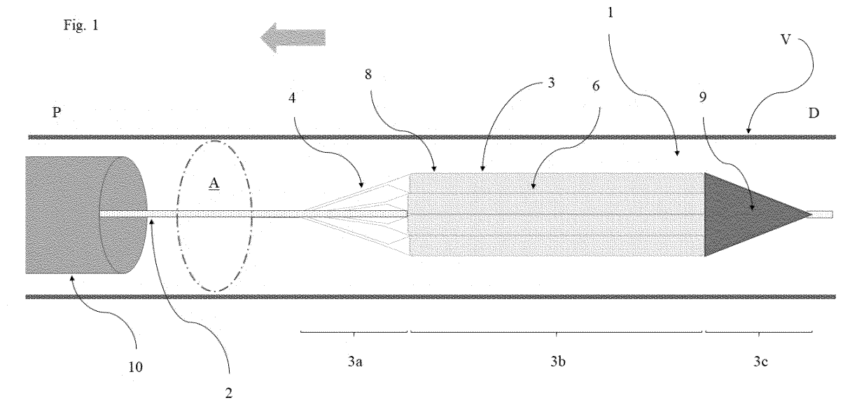

With reference to FIG. 1, there is illustrated catheter device 1 for extracting an occlusion A from a blood vessel V, said device 1 comprising a shaft 2 having a proximal end and a distal end; and an expandable structure 3 having a proximal portion 3a and a distal portion 3c and coupled to or adjacent the distal end of the shaft 2; said the expandable structure 3 being configured to expand radially around the shaft; wherein the proximal portion of the expandable structure 3 comprises one or more fragmentors 4 for fragmenting the occlusion A.

In the accompanying figures, “proximal” is designated by “P” and “distal is designated by “D”.

Still with reference to FIG. 1, the expandable structure 3 comprises a proximal portion 3a, an intermediary portion 3b and a distal portion 3c.

The proximal portion 3c is coupled to the expandable structure 3 to the shaft 2. For example, the proximal portion 3a comprises a plurality of tethers 5. Each tether comprises a proximal end and a distal end. The proximal ends of the tethers 5 are coupled to the shaft 2. The distal ends of the tethers 5 may be coupled to the intermediary portion 3b, or to the distal portion 3c if the expandable structure 3 does not comprise an intermediary portion 3b.

The proximal portion 3c comprises one or more fragmentors 4. Examples of fragmentors 4 are cutters, such as one or more blades. The fragmentors 4 may be coupled to the proximal portion 3c, or may be integrally formed with the proximal portion 3c.

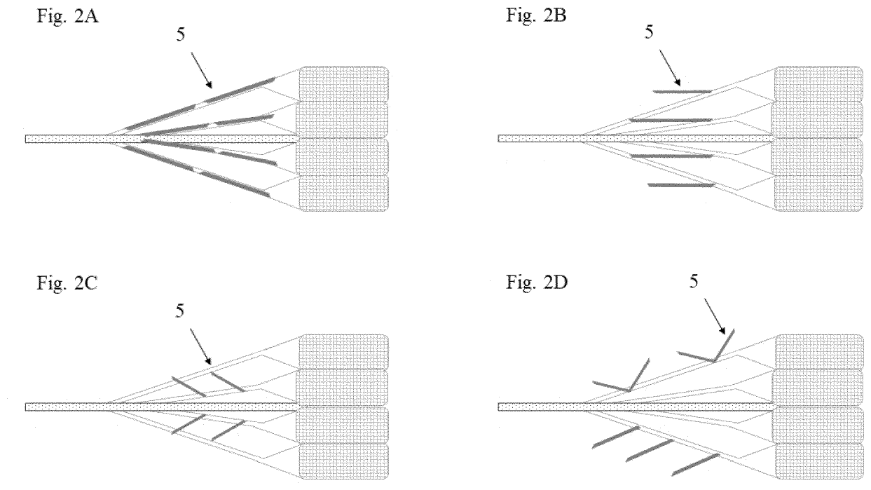

With reference to FIG. 2a, the fragmentors 4, in this case blades, are aligned with the tethers 5, to allow direct cutting efficiency upon pulling the device 1 from a distal to a proximal direction. The fragmentors 4 may be configured and arranged in a profile corresponding to the contour of the proximal portion 3c. In a preferred embodiment, the proximal portion 3c is tapered or substantially conical to facilitate penetration into, and hence fragmentation of, the occlusion A.

The fragmentors 4 may be substantially parallel to the shaft 2 (as illustrated for example in FIG. 2b), and/or they may be preferentially oriented or angled to guide the resulting fragments towards a desired direction (as illustrated for example in FIGS. 2c and 2d).

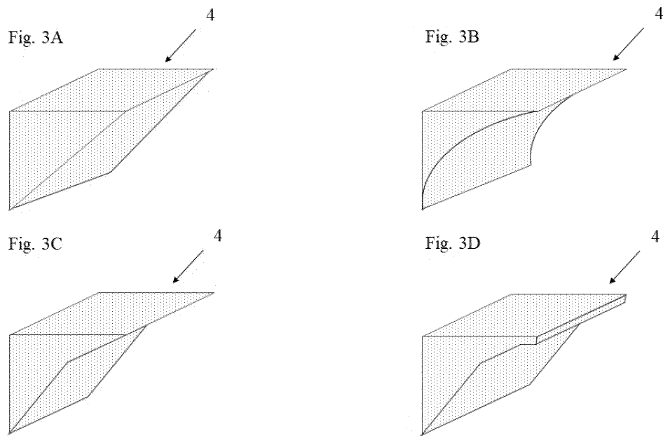

The fragmentors 4 may comprise a preferential profile, which may be obtained by laser-cutting (for example by high-precision laser-cutting) of the proximal portion 3c. With reference to FIGS. 3a to 3b, individual tethers 5 may be shaped to comprise one or more chamfers. FIG. 3a illustrates a single straight chamfer. FIG. 3b illustrates a single curved chamfer. FIG. 3c illustrates a double-angle chamfer. FIG. 3d illustrates a double-chamfer with an additional lead. It is envisaged that the double-chamfer may be replaced with a curved chamfer, and vice versa.

The intermediate portion 3b of the expandable structure 3 comprises a plurality of inflatable balloons 6 arranged circumferentially around a lumen 7 of the expandable structure 3. The lumen 7 allows for fluid flow and for passage of occlusion fragments and emboli therethrough, when the expandable structure 3 is in an expanded (working) configuration. An inflation line (not shown) may be provided to inflate the inflatable balloons 6 into a working configuration.

The intermediate portion 3b comprises an expandable scaffold (not shown), for example made of a substantially rigid material. The expandable scaffold is folded (for example folded around the shaft 2) in a delivery and/or retrieval configuration, and deployed radially outwards relative to the shaft 2 into a working configuration.

The expandable scaffold assists in the folding and deployment of the expandable structure 3. The expandable scaffold provides structural support to the inflatable balloons. The expandable scaffold positions the inflatable balloons 6 around a lumen 7 in a working configuration.

The intermediate portion 3 may comprise one or more cover membranes 8. The membrane 8 may be made of or comprise fibers, for example a high tensile strength fiber-based material. In an embodiment, the inflatable balloons 6 are made of or comprise a membrane. The membrane 8 may cover the plurality of balloons 6.

In an embodiment, the catheter device 1 does not comprise inflatable balloons 6. Instead, the device 1 comprises an expandable scaffold, which may comprise a cover membrane 8. The cover membrane 8 preferably covers the outer circumference of the expandable scaffold. This configuration is particularly advantageous in that a low-profile may be achieved in the folded configuration (e.g. in the delivery stage, the occlusion penetration stage and/or the device and clot retrieval stage).

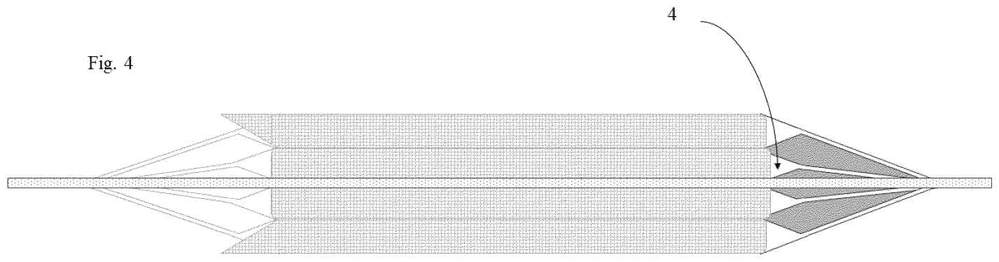

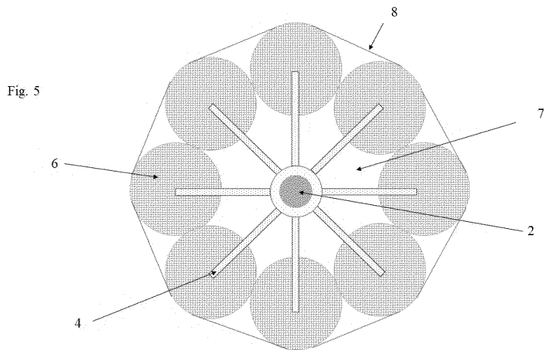

The proximal end of intermediate portion 3b of the expandable structure 3 may comprise guiding elements for guiding emboli and fragments towards the lumen 7 of the expandable structure 3. As illustrated for example in FIG. 4, the mouth (at the proximal end) of intermediate portion 3b may comprise a chamfered inner surface, guiding occlusion fragments towards the centre of the lumen to facilitate collection of the fragments.

The distal portion 3c of the expandable structure 3 comprises securing or attachment elements for coupling the expandable structure 3 to the shaft 2. For example, the distal portion 3c may comprise a plurality of tethers 5. Each tether comprises a proximal end and a distal end. The distal ends of the tethers 5 are coupled to the shaft 2. The proximal ends of the tethers 5 may be coupled to the distal end of the intermediary portion 3b, or to the proximal portion 3a if the expandable structure 3 does not comprise an intermediary portion 3b.

The distal portion 3c preferably comprises a collection element 9 for collecting emboli and/or fragments, for example in the form of a basket. The collection element 9 may comprise a fluid permeable material or structure, so that the fragments are retained therein, but fluid (blood) may flow therethrough. Preferred materials include, but are not limited to fiber materials (e.g. high tensile strength fiber-based material, UHMWPE) and structures, polymeric materials and structures, and metallic materials and structures (e.g. mesh or net).

In use, a delivery sheath or catheter 10 may be inserted in a conventional manner in a patient's artery and advanced through the patient's circulatory system to the blood vessel V obstructed by an occlusion A, until the distal end of the catheter 10 is adjacent the occlusion A.

The catheter device 1 is inserted into the catheter 10, with the expandable structure 3 in a folded configuration. For example, the structure 3 is folded against and around the shaft 2 in a low profile insertion/delivery configuration. The device 1 is advanced along the catheter 10, until the distal end of the catheter 10 is adjacent the occlusion A.

The expandable structure 3 is advanced further to penetrate the occlusion. As the expandable structure 3 is still in a folded configuration during this step, and owing to the low-profile of the structure 3, the production of loose fragments is minimised. The distal end of the expandable structure 3 may comprise a coring structure to further facilitate the advancement of the structure 3 through the occlusion A.

The expandable structure 3 is advanced until its proximal portion 3c has partially or completely exited the occlusion A, and is positioned distally from the occlusion A.

The expandable structure 3 is deployed into a working position. The deployment may be effected by expanding the expandable scaffold and/or inflating the inflatable balloons 6. In the expanded configuration, the expandable structure 3 defines a lumen 7 for fluid flow and fragment passage therethrough. The expandable structure 3 may be configured and arranged so as to minimise the passage of fragments across the structure itself. For example, the expandable structure 3 comprises a mesh, net and/or a cover membrane surrounding the lumen 3 and or the intermediate portion 3b of the expandable structure 3.

In the fragmentation step, the catheter device 1 is pulled back towards the distal end of the occlusion A. In the deployed configuration, the fragmentors 4 cut, shear, break or otherwise fragment the occlusion A into emboli.

The expandable structure 3 may trawl the emboli and occlusion fragments. The emboli and fragments may also be guided towards the lumen 7 by oriented or angled fragmentors 4 and/or by a chamfered surface at or adjacent the proximal opening of the intermediate portion 3b of the expandable structure 3.

The emboli and fragments may travel along the lumen 7 and be caught in a collection element 9 (for example a collection basket) in the distal portion 3c of the expandable structure 3. The collection element 9 may comprise a material and/or structure capable of retaining emboli and fragments, but allowing blood flow therethrough.

Once the occlusion A has been broken down into fragments sufficiently small for extraction, the catheter device 1 is pulled towards the distal end of the catheter 10.

One or more capturing element may be used to capture or trap fragments as the catheter device 1 approaches the catheter 10 for retrieval. For example, the capturing element may be configured and arranged to cover the proximal portion 3a of the expandable structure 3 or to cover the collection element 9 in which the fragments have gathered. The capturing element may be tapered or conical, or have an inner contour substantially corresponding to the outer contour of the proximal portion 3a. The capturing element may be folded in a delivery configuration and deployed in a capturing/working configuration. The capturing element may assist in the folding of the expandable structure 3 into the catheter 10 in the retrieval step.

As the proximal portion 3a contacts and advances into the catheter 10, the expandable structure 3 may be folded into a retrieval configuration. As the expandable structure 3 is collapsed, the fragmented occlusion A is trapped in the lumen 7, in the collection element 9 and/or in the capturing element. The catheter device 1 may be retrieved through the catheter 10.

Thus, from the above description, it can be seen that the present disclosure provides a device and a method for an efficient fragmentation and extraction of an occlusion from a vessel.

All of the above are fully within the scope of the present disclosure and are considered to form the basis for alternative embodiments in which one or more combinations of the above described features are applied, without limitation to the specific combination disclosed above.

In light of this, there will be many alternatives which implement the teaching of the present disclosure. It is expected that one skilled in the art will be able to modify and adapt the above disclosure to suit its own circumstances and requirements within the scope of the present disclosure, while retaining some or all technical effects of the same, either disclosed or derivable from the above, in light of his common general knowledge in this art. All such equivalents, modifications or adaptations fall within the scope of the present disclosure.

1. A catheter device for extracting occlusion from a blood vessel, said device comprising:
a shaft having a proximal end and a distal end; and
an expandable structure having a proximal portion and a distal portion and coupled to or adjacent the distal end of the shaft, the expandable structure being configured to expand radially around the shaft; and
a lumen for fluid flow and fragment passage, wherein the inflatable balloons are positioned circumferentially around the lumen;
wherein the proximal portion of the expandable structure comprises one or more fragmentors.

2. The catheter device according to claim 1, wherein the proximal portion is tapered or substantially conical.

3. The catheter device according to claim 1, wherein the proximal portion comprises a plurality of tethers.

4. The catheter device according to claim 1, wherein the one or more fragmentors comprise cutters, such as one or more blades.

5. The catheter device according to claim 1, wherein the one or more fragmentors are aligned with the proximal portion of the expandable structure

6. The catheter device according to claim 1, further comprising a lumen for fluid flow and fragment passage, and wherein the one or more fragmentors are configured and arranged to guide occlusion fragments towards said lumen.

7. The catheter device according to claim 1, wherein the expandable structure comprises an expandable scaffold.

8-9. (canceled)

10. The catheter device according to claim 1, wherein the device comprises a lumen for fluid flow and fragment passage and a guiding structure for guiding fragments towards the lumen.

11. The catheter device according to claim 1, wherein the distal portion of the expandable structure comprises a collecting element for collecting the occlusion fragments.

12. The catheter device according to claim 11, wherein the collecting element is configured to allow fluid flow therethrough.

13. A catheter method for extracting an occlusion from a vessel, comprising the steps of:
advancing a catheter along the vessel;
advancing a device according to claim 1 through the catheter;
advancing the device through the occlusion until the proximal portion of the device protrudes partially or completely beyond the occlusion;
expanding the expandable structure; and
retrieving the device so as to fragment the occlusion.

14. The catheter method according to claim 13, wherein the device is advanced through the catheter or through the occlusion, with the expandable structure is a non-expanded configuration.

15. The catheter method according to claim 13, wherein the step of expanding expandable structure comprises expanding a scaffold.

16. The catheter method according to claim 13, wherein the step of expanding expandable structure comprises inflating a plurality of inflatable balloons.

17. The catheter method according to claim 13, wherein the device comprises a lumen for fluid flow between the proximal portion and the distal portion, and the method comprising the step of guiding the occlusion fragments towards said lumen.

18. The catheter method according to claim 13, further comprising the step of collecting the occlusion fragments.

19. The catheter method according to claim 13, comprising the step of retrieving the device through the occlusion or through the catheter, with the expandable structure is a non-expanded configuration.

20. A catheter system comprising a catheter and a device according to claim 1.

21. The catheter system according to claim 20, wherein the system further comprises a capturing element for capturing the occlusion fragments.

22. A catheter device for extracting an occlusion from a blood vessel, said device comprising:
a shaft having a proximal end and a distal end; and
an expandable balloon having a proximal portion and a distal portion and coupled to or adjacent the distal end of the shaft; the expandable balloon being configured to expand radially around the shaft;
wherein the proximal portion of the expandable balloon comprises one or more cutters.
